(19) United States
(12) Patent Application Publication
(10) Pub. No.: US 2007/0281321 A1
(43) Pub. Date: Dec. 6, 2007

(54) BIOSENSOR FOR MEASUREMENT OF SPECIES IN A BODY FLUID

(75) Inventors: Milind P. Nagale, Lowell, MA (US); Paul Gamache, Hudson, NH (US); Ian Acworth, Melrose, MA (US); William J. Scott, Billerica, MA (US); Eric W Zink, Burlington, MA (US); Mark L. Bowers, Arlington, MA (US); Michael Granger, Chandler, AZ (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: ESA BIOSCIENCES, INC., Chelmsford, MA (US)

(21) Appl. No.: 11/756,040

(22) Filed: May 31, 2007

Related U.S. Application Data
(60) Provisional application No. 60/809,619, filed on May 31, 2006.

(51) Int. Cl. G01N 33/53; C12Q 1/26; C12M 3/00

(52) U.S. Cl. 435/7.1; 435/25; 435/287.2

(57) ABSTRACT

Certain embodiments disclosed herein are directed to devices configured to detect the level of a biomarker in a body fluid. In some examples, the device includes two or more electrodes for electrochemical detection of the biomarker in the body fluid. Methods of using the device are also disclosed.

### PRIORITY APPLICATION

This application claims the benefit of U.S. Provisional Application No. 60/809,619 filed on May 31, 2006, the entire disclosure of which is hereby incorporated herein by reference for all purposes.

### FIELD OF THE TECHNOLOGY

Certain examples of the technology described herein are directed to devices and methods for measuring species in a biological fluid. More particularly, in certain embodiments, an apparatus for measuring levels of compounds in various body fluids using electrochemical detection is described.

### BACKGROUND

Diagnosis of diseases in a rapid and a cost efficient manner is difficult for many diseases. Early detection of disease states may provide for increased treatment options and enhanced survival rates. There remains a need for better devices and methods to detect disease states.

### SUMMARY

In accordance with a first aspect, a device comprising a support, a first electrode, a second electrode and a chamber is provided. In certain examples, the first electrode and the second electrode each may be disposed on the support. In other examples, the chamber may be disposed on the support and include a sample area configured to receive a biomarker and a biological recognition element specific for the biomarker, the chamber being in fluid communication with at least one of the first electrode and the second electrode. In some examples, at least one of the first and second electrodes may further include, or be electrically coupled to, a detector.

In some examples, the biological recognition element may be an oxidoreductase. In other examples, the biomarker may be a substrate and the biological recognition element may be an enzyme specific for the substrate. In some examples, the device may also include a third electrode electrically coupled to the detector. In certain examples, the detector may be electrochemical detector.

In accordance with another aspect, a device comprising a support and a biological recognition element disposed on the support is disclosed. In certain examples, the biological recognition element may be effective to produce an electrochemically detectable reaction product from a body fluid comprising one or more biomarkers indicative of a disease state is provided. In some examples, the biological recognition element may be selected from the group consisting of an enzyme, an antibody and an antigen. In other examples, the biological recognition element may be an oxidoreductase. In certain examples, the device may also comprise at least one electrode for detecting the electrochemically detectable reaction product. In other examples, the device may further comprise an electrode array for detecting the electrochemically detectable reaction product. In certain examples, the device may be configured to detect the electrochemically detectable reaction product when the biomarker is present above a threshold value in the body fluid.

In accordance with an additional aspect, a point of care device for detecting a biomarker indicative of a disease state is provided. In certain examples, the device may be configured to receive a body fluid and may comprise a biological recognition element effective to convert a biomarker in the body fluid into an electrochemically detectable reaction product. In some examples, the biological recognition element may be an oxidoreductase. In other examples, the device may further comprise an electrochemical detector for detecting the electrochemically detectable reaction product. In some examples, the electrochemical detector may be configured for potentiometric, coulometric or charged aerosol detection.

In accordance with another aspect, a method of detecting a biomarker in a body fluid is disclosed. In certain examples, the method comprises exposing the biomarker to a biological recognition element disposed in a device comprising at least one electrode. In some examples, the method further comprises detecting a reaction product after conversion of the biomarker into the reaction product by the biological recognition element. In certain examples, the detecting step comprises electrochemically detecting the reaction product. In other examples, the method may further comprise detecting a second reaction product after conversion of a second biomarker in the body fluid into the second reaction product by a second biological recognition element disposed in the device.

Additional aspects, features and details of the technology disclosed herein are discussed in more detail below.

### BRIEF DESCRIPTION OF FIGURES

Certain illustrative embodiments are described in more detail below with reference to the accompanying figures in which:

FIG. 1 is a schematic of a two electrode device, in accordance with certain embodiments;

FIG. 2 is a schematic of a three electrode device, in accordance with certain embodiments;

FIG. 3 is a schematic of the three electrode device of FIG. 2 with an active reagent disposed on a working electrode, in accordance with certain embodiments;

FIG. 4 is a schematic of the three electrode device of FIG. 3 with an insulating layer disposed on a support, in accordance with certain embodiments;

FIG. 5 is a schematic of the three electrode device of FIG. 4 with an additional insulating layer disposed on the device, in accordance with certain embodiments;

FIG. 6 is a schematic of the three electrode device of FIG. 5 with a protective layer disposed on the device, in accordance with certain embodiments; and

FIG. 7 is a schematic of the three electrode device of FIG. 6 showing a sample introduced into the device, in accordance with certain embodiments;

FIG. 8 shows chromatograms indicating the detection of choline by electrochemical detection after separation by LC.

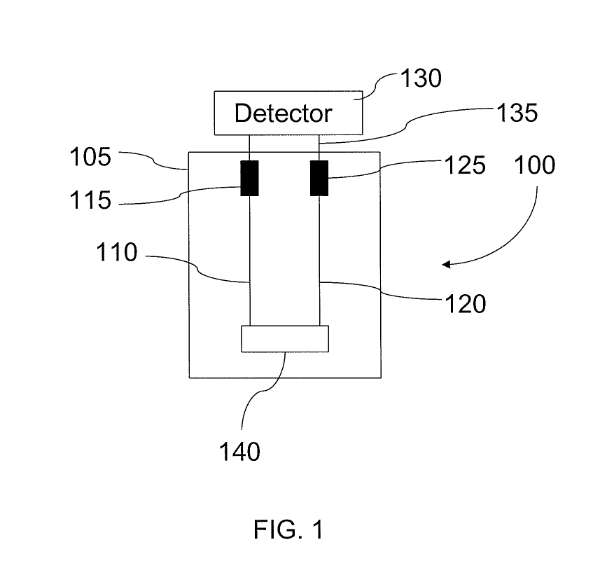

It will be recognized by the person of ordinary skill in the art, given the benefit of this disclosure, that the certain features shown in FIGS. 1-7 are not necessarily drawn to scale. The dimensions and characteristics of some features in the figures may have been enlarged, distorted or altered relative to other features in the figures to facilitate a better understanding of the illustrative examples disclosed herein.

### DETAILED DESCRIPTION

It will be recognized by the person of ordinary skill in the art, given the benefit of this disclosure, that the devices and methods disclosed herein represent a significant development in devices and methods for detecting and/or predicting disease states. Devices configured for detection of biomarkers can be produced, for example, at low cost, with high reproducibility and for use as point of care devices. The devices disclosed herein may be used, for example, in an amperometric or potentiometric mode depending on the chemistry applied to the working electrode.

In accordance with certain examples, the devices and methods disclosed herein may be configured to detect one or more biomarkers in a body fluid. The device may be configured in cartridge form with an “on-board” detector such that it may be used without any additional equipment or devices, or it may be configured to interface with other devices or equipment such as, for example, electrochemical detectors or light absorption or emission detectors. In some examples, the devices may be configured such that indicia are provided if the level of biomarker exceeds a threshold value. Such indicia include, but are not limited to, switching on of a light, beeping, flashing lights or the like. In other examples, the device may output the detected level of the biomarker. In yet other examples, the device may be configured such that no result is provided unless the level of biomarker in a body fluid exceeds a threshold value. Additional advantages and configurations of the device are discussed in more detail below.

A number of useful biomarkers in body fluids such as blood are specific substrates of various oxidoreductases. For example, the following chemical compounds present in many biological systems have an oxidoreductase enzyme that can act upon them in a more or less specific manner. This list includes a number of substrates including, but not limited to, alcohol, ascorbate, bilirubin, choline, galactose, glutamate, gulonolactone, lactate, lysine, pyruvate, tyramine and xanthine.

Many of the oxidoreductase enzyme substrates have been shown to be biomarkers of a disease state or disorder. For example, measurement of whole blood choline (WBCHO) and plasma choline (PLCHO)—choline being a substrate of choline oxidase—was identified as one of nine potential future biomarkers for detection of ischemia and risk stratification in acute coronary syndrome (ACS) in a review article written on behalf of the Committee on Standardization of Markers of Cardiac Damage of the International Federation of Clinical Chemistry which appeared in the journal Clinical Chemistry. See Apple F S, Wu A H, Mair J, Ravkilde J, Panteghini M, Tate J, et al. Future biomarkers for detection of ischemia and risk stratification in acute coronary syndrome. Clin. Chem. 2005; 51:810-24. Currently there is no point-of-care (POC) diagnostic device available commercially for choline measurement.

Biochemical studies have been performed that correlate rapidly increasing levels of WBCHO and PLCHO with stimulation of phospholipase D enzyme activation and other signal transduction processes that are thought to be fundamental to coronary plaque destabilization and tissue ischemia. See: Wu A H, Markers for early detection of cardiac diseases, Scand. J. Clin. Lab. Invest. Suppl., 2005; 240:112-21. A study of 327 patients with suspected ACS showed that “WBCHO was a significant predictor of cardiac death or cardiac arrest, life-threatening cardiac arrhythmias, heart failure, and coronary angioplasty when measured in the first blood sample on admission.” See: Danne O, Mockel M, Lueders C, Mugge C, Zschunke G A, Lufft H, et al., Prognostic implications of elevated whole blood choline levels in acute coronary syndromes, Am. J. Cardiol., 2003; 91:1060-7. This study appears to be the only study that specifically evaluates the clinical relevance of WBCHO or PLCHO measurements in a significant patient population. In this study, “cardiac troponins and WBCHO were the most powerful independent predictors in multivariate analysis, and the combination of WBCHO and cardiac troponins allowed a superior risk assessment compared with each test alone.” The review article states “when interpreting results for individual patients, it is useful to have both WBCHO and PLCHO data to identify risks . . . to target advanced treatment strategies . . . ” The article also states “Development of rapid POC tests and central laboratory assays of WBCHO and PLCHO will be necessary to evaluate whether these markers will help to identify such high-risk patients in clinical practice.”

Measurements using liquid chromatography with electrochemical detection (LC-EC) that are primarily geared toward the study of acetylcholine (Ach) neurotransmission in tissue and microdialysis perfusates have been performed. See: Greaney M D, Marshall D L, Bailey B A, Acworth I N, Improved method for the routine analysis of acetylcholine release in vivo: quantitation in the presence and absence of esterase inhibitor, J. Chromatogr., 1993; 622:125-35. This methodology involved chromatographic separation of Ach and CHO, specific conversion of these analytes using on-line immobilized enzymes, and measurement of the reaction by-product, hydrogen peroxide, using an EC cell with a Pt working electrode. Ach and choline (CHO) are therefore not directly detected by EC; rather measurement of their concentrations is derived indirectly via conversion to the EC-active molecule, hydrogen peroxide. The methodologies used for LC-EC determination of choline can be used in the devices disclosed herein by combining a biological recognition element (such as an enzyme with specificity towards CHO) with an electrochemical cell and a detector. With appropriate reagent and sensor design, separation of CHO from other components is not necessary for detection of CHO levels in body fluids thus making it feasible to design a POC device. The device may be designed to accommodate most body fluids (e.g., blood, plasma, serum, cerebrospinal fluid, saliva, tears, exhalation vapor, lung lavage, sperm, urine etc.) and may be capable of monitoring both extracellular and intracellular analyte levels.

In accordance with certain examples, a device comprising at least two electrodes (working and a reference) for use in detecting a biomarker is disclosed. In certain examples, the electrodes may be placed on an insulating support and provided with a region for electrical contact to a detector. For example and referring to FIG. 1, device 100 includes a support 105, a first electrode 110 and a second electrode 120. Each of the electrodes 110 and 120 may be electrically coupled to a detector 130 through an interconnect or electrical lead, such as lead 135. Electrode 110 has an electrical contact 115, and electrode 120 has a contact 125. Each of contacts 115 and 125 may be used to provide an electrical signal to the detector 130. Each of the electrodes 110 and 120 may also be electrically coupled to a chamber 140. Fluid to be tested may be supplied to the chamber 140 using suitable devices and methods such as, for example, those discussed herein.

In accordance with certain examples, the support 105 used in the devices disclosed herein may vary in composition and size. Illustrative materials for use in the support include, but are not limited to polymers such as, for example, polyvinyl chloride (PVC), polycarbonate, polyester and the like. In certain examples, the support 105 may include fillers, fibers, particles and the like to provide structural reinforcement to the support and/or to increase the rigidity of the support. In some examples, the materials used in the support 105 may act as an insulator. The insulator may prevent loss of electrical currents and may act to maintain the temperature of the device at a desired temperature, e.g., 37° C., during detection. In certain examples, the support has dimensions of about 4-5 cm long, e.g., about 4.5 cm long, by about 1-2 cm wide, e.g., about 1.5 cm wide, and is about 0.025 to 1 cm thick, e.g., about 0.05 cm thick. Additional materials and dimensions for the devices disclosed herein will be readily selected by the person of ordinary skill in the art, given the benefit of this disclosure.

In accordance with certain examples, each of the electrodes 110 and 120 of the devices disclosed herein may be produced using a conductive material. For example, materials such as platinum, carbon, gold, silver, iridium, boron doped diamond, etc. may be used in the electrodes disclosed herein. The conductive material may be coated or plated on a nonconductive material to provide an electrode, or the conductive material itself may be used as an electrode. The size of the electrodes may depend on numerous factors such as, for example, the methods used to dispose the electrodes onto the support, the sample volume required for analysis and the like. In certain examples, the electrodes each may be about 1 cm wide to about 1 cm long. The exact shape or cross-sectional outline of each electrode may vary, and in certain examples the electrodes each may be cylindrical, circular, plate-like, have a circular cross-section, or may take other forms and configurations. The electrodes may also be configured into various arrays and ensembles. The precise array or ensemble arrangement may vary in terms of layout, shape, size and number. The electrode arrays can be fabricated using micro fabrication methods such as MEMS (micro-electro-mechanical systems) techniques. Microelectrode arrays may be produced where each active electrode has dimensions on the order of a few μm or smaller. Such microelectrodes may have the added benefit of improving the sensitivity of the biosensor as well as reducing deleterious effects such as electrode fouling which can degrade the performance of the biosensor. It will be within the ability of the person of ordinary skill in the art, given the benefit of this disclosure, to select other materials, dimensions and shapes for designing suitable electrodes for use in the devices disclosed herein.

In accordance with certain examples, various methods may be used to pattern an electrode on the support. For example, screen-printing, vapor deposition, sputtering, laser ablation, electroplating and combinations thereof may be used to pattern an electrode on the support. In some examples, an electrode may be patterned or disposed directly on the support, whereas in other examples an electrode may be produced separately from the support and transferred to the support post-production. Other methods of electrode fabrication and patterning may be accomplished by photo-lithographic means, micromachining, electro discharge machining (EDM) and various methods of chemical etching. Ensemble electrodes may be fabricated by inserting electrode elements (such as fibers) in an insulating matrix (such as an epoxy resin or a polymer). Additional methods of producing an electrode useful in the devices disclosed herein will be readily selected by the person of ordinary skill in the art, given the benefit of this disclosure.

In accordance with certain examples, the detector 130 of the devices disclosed herein is typically selected based on the species to be detected. In the case where the species to be detected is electrochemically active, or can be rendered electrochemically active, an electrochemical detector, such as, for example, an amperometric, potentiometric or coulometric detector may be used. In some examples, corona aerosol detection may be performed. In certain examples, two or more detectors may be used. For example, in the case where the species to be detected absorbs visible or ultraviolet light, a UV/Visible absorption detector may be used, either alone or in combination with an electrochemical detector. In the case where the species is fluorescent or phosphorescent, fluorescence or phosphorescence emission may be measured after the species is excited. Additional types of detectors for detection of a particular species will be selected by the person of ordinary skill in the art, given the benefit of this disclosure.

In certain examples, the detector 130 may be omitted from the device 100, and the device 100 may interface with a separate detector located off-board. For example, device 100 may be inserted into or fluidly connected a detector, such as commercially available spectrometers, spectrophotometers and electrochemical detectors, such that reaction product produced in the device may be provided to the detector for detection. In some examples, the reaction product may be provided from the device 100 to a detector through one or more outlet ports that couples fluid from the device 100 to a fluid channel of the detector. For example, the device 100 may be plugged or inserted into a liquid chromatograph such that species in the device 100 may be separated followed by subsequent detection. In some examples, reaction product may be off-loaded from the device 100 manually by an operator using a syringe or other suitable device that may remove fluid from the device 100. The off-loaded reaction product may then be introduced into a suitable detector to identify various species in the reaction product. It will be within the ability of the person of ordinary skill in the art, given the benefit of this disclosure, to couple the devices disclosed herein to one or more detectors.

In embodiments where the detector is configured for electrochemical detection, a desired potential or current is typically applied to the electrodes for a pre-determined amount of time. The current or potential may be monitored at the working electrode. The monitored current or potential may be converted to a biomarker concentration or level based on calibration information provided to the detector or using a lookup table stored on a memory chip in the detector or on a memory chip included on the device. The level of biomarker may be displayed on a screen, outputted to a printer or to an electronic device such as, for example, a personal digital assistant, or otherwise sent to a desired location electronically by wired or wireless means. In embodiments where the device is configured to detect biomarker above a threshold level, if the level of biomarker in a body fluid is below a threshold level, then a message indicating that the level is below a threshold level may be sent or displayed. The detector may also store the results optionally with date and/or time stamps. The detector may include one or more electronic interfaces for transferring the results to another electronic device. Additional features that may be included on the detector will be readily selected by the person of ordinary skill in the art, given the benefit of this disclosure.

In accordance with certain examples, the chamber 140 may be configured to receive a sample or a sample mixture. In certain examples, the chamber 140 may be constructed and arranged to receive a sample as well as a reagent or reagent mixture. For example, the chamber 140 may receive blood from a patient that contains a biomarker. The chamber 140 may also receive a buffer, an enzyme, a solution or the like that may be used to detect the presence and/or level of the biomarker in the blood sample from the patient. In some examples, the chamber may be sized and arranged to receive blood from a patient's finger after the patient pricks his or her finger with a needle. For example, the patient may prick their finger and then insert their finger into the device. The chamber receives blood from the patient's finger, and the blood can be subsequently analyzed for a particular biomarker of interest. In other examples, a body fluid other than blood, e.g., urine, saliva, bile, cerebrospinal fluid, mucus secretions, lymph, sputum, etc. may be used. It will be within the ability of the person of ordinary skill in the art, given the benefit of this disclosure, to select suitable body fluids and the methods to obtain the fluids for use with the devices disclosed herein.

In certain examples, the chamber 140 may include a biological recognition element selected for a particular biomarker. In certain examples, the biological recognition element may be an enzyme having high specificity for the biomarker. In some examples, the biomarker acts as a substrate for the enzyme in which case the product from the enzymatic reaction is detected. One particular class of biological recognition element is an oxidoreductase enzyme that produces hydrogen peroxide concomitantly with the selective oxidation of its biomarker substrate. A specific oxidoreductase-biomarker pair of interest is choline oxidase and choline. The choline oxidase oxidizes choline, in the presence of oxygen, to betaine aldehyde and hydrogen peroxide. The amount of hydrogen peroxide that is produced is proportional to the amount of choline present in the sample. By electrochemically detecting the level of hydrogen peroxide present, the level of choline in the sample may be determined.

In accordance with certain examples, many different biological recognition elements may be used in the devices and methods disclosed herein. Exemplary biological recognition elements include proteins, such as antibodies, enzymes, antigens and the like, amino acids, lipids, carbohydrates, steroids, nucleotides, and the like. One particular class of biological recognition elements that are particularly useful in the devices disclosed herein are oxidoreductase enzymes. Illustrative oxidoreductase enzymes and their substrate(s) (shown in parenthesis below) include, but are not limited to, those classified as ECI oxidoreductases by the Nomenclature Committee of the International Union of Biochemistry and Molecular Biology (NC-IUBMB), e.g., oxygen acceptor oxidoreductases in family EC 1.1.3 such as malate oxidase ((S)-malate), glucose oxidase (β-D-glucose), hexose oxidase (D-glucose and other hexoses), cholesterol oxidase (cholesterol), aryl-alcohol oxidase (aromatic primary alcohols), L-gulonolactone oxidase (L-gulono-1,4-lactone or ascorbate), galactose oxidase (D-galactose), pyranose oxidase (D-glucose), L-sorbose oxidase (L-sorbose), pyridoxine 4-oxidase (pyridoxine), alcohol oxidase (primary alcohols), (S)-2-hydroxy-acid oxidase ((S)-2-hydroxy acid), ecdysone oxidase (ecdysone), choline oxidase (choline), secondary-alcohol oxidase (secondary alcohols), 4-hydroxymandelate oxidase ((S)-2-hydroxy-2-(4-hydroxyphenyl)acetate), glycerol-3-phosphate oxidase (sn-glycerol 3-phosphate), thiamin oxidase (thiamine), hydroxyphytanate oxidase (L-2-hydroxyphytanate), N-acylhexosamine oxidase (N-acetyl-D-glucosamine), polyvinyl-alcohol oxidase (polyvinyl alcohol), D-arabinono-1,4-lactone oxidase (D-arabinono-1,4-lactone), vanillyl-alcohol oxidase (vanillyl alcohol), H₂O forming nucleoside oxidase (adenosine and 5′-dehydroadenosine), D-mannitol oxidase (mannitol) and xylitol oxidase (xylitol). Other illustrative oxidoreductases and their substrates (in parentheses) include, but are not limited to, xanthine oxidase (xanthine), L-galactonolactone oxidase (L-galactonolactone), dihydroorotate oxidase ((S)-dihydroorotate), coproporphyrinogen oxidase (coproporphyrinogen III), protoporphyrinogen oxidase (protoporphyrinogen IX), bilirubin oxidase (bilirubin), acyl-CoA oxidase (acyl-CoA), dihydrouracil oxidase (5,6-dihydrouracil), tetrahydroberberine oxidase ((S)-tetrahydroberberine), secologanin synthase (loganin), tryptophan α,β-oxidase (L-tryptophan), aldehyde oxidase (aldehydes), pyruvate oxidase (pyruvate), oxalate oxidase (oxalate), glyoxylate oxidase (glyoxylate), CoA-acetylating pyruvate oxidase (pyruvate+CoA), indole-3-acetaldehyde oxidase (indole-3-acetaldehyde), pyridoxal oxidase (pyridoxal), aryl-aldehyde oxidase (aromatic aldehydes), retinal oxidase (retinal), and 4-hydroxyphenylpyruvate oxidase (4-hydroxyphenylpyruvate). Additional suitable oxidoreductases include those that use one or more of oxygen, NAD⁺, NADP⁺, a cytochrome, a disulfide, a quinone, and an iron-sulfur protein as an acceptor. Additional suitable oxidoreductases and other enzymes for use in the devices and methods disclosed herein will be readily selected by the person of ordinary skill in the art, given the benefit of this disclosure.

In other examples, the chamber 140 may be designed to receive a test strip that includes a biological recognition element. The exact configuration of the test strip may vary. In some examples, the test strip may be sized and arranged to be inserted into a slot of the device such that at least a portion of the test strip is in fluid communication with the chamber 140. In other examples, the entire test strip may be inserted into the chamber 140 and a buffer or solution is provided to the chamber such that the sample can be detected. In some examples, the biological recognition element disposed on the test strip may be reconstituted in the device by placing the test strip in a buffer or solution.

In accordance with certain examples, the exact configuration and dimensions of the overall device may vary. In embodiments where the device is configured for home use, the device may take the form of a cartridge or the like that includes all elements, e.g., electrodes, detector, biological recognition element, etc. In embodiments where the device is intended for use in a clinical setting, the device may be configured to receive one or more test strips containing a patient sample. The test strips may include, for example, a biological recognition element for a particular biomarker and may be designed for use with a single sample. The device itself, however, may be used numerous times. In some embodiments designed for the clinical setting, the entire device may be configured as a single use device, e.g., a cartridge, that can receive a patient sample and rapidly provide for detection of a particular biomarker in the patient sample. Additional configurations for the devices disclosed herein will be readily selected by the person of ordinary skill in the art, given the benefit of this disclosure.

In accordance with certain examples, the devices disclosed herein may include one or more ports for providing buffers, solutions, and the like to the device. In certain examples, the port may be configured to receive fluid from a reservoir. In other examples, the port may be configured to receive a sample from a patient. Other functions of a port for use with the devices disclosed herein will be readily selected by the person of ordinary skill in the art, given the benefit of this disclosure.

In accordance with certain examples, the devices disclosed herein may be used to measure or detect a biomarker present in a patient sample. In certain examples, the electrodes of the device are in fluid communication with a reagent mixture consisting of a sample and a biological recognition element, e.g., choline oxidase or choline dehydrogenase. The reagent mixture may further include electrochemical mediators, buffers, salts, ions, detergents, wetting agents or other species that may be useful in promoting a reaction between the biomarker and the biological recognition element.

In accordance with certain examples, two or more of biosensors may be combined into a single device, e.g., for use in a multiplex mode. In certain examples, a single biosensor device may include a plurality of working electrodes each being able to detect a different biomarker at the same time or in quick succession. In certain embodiments, the biomarker detecting working electrodes may share a common sample inlet port, channel, reference and auxiliary electrodes and other components of the biosensor device such as buffers, solutions, reservoirs and the like. In other examples, a device may include separate ports, channels, biomarker sensing working electrodes, reference and auxiliary electrodes so that two different types of sample could be examined, e.g., simultaneously or in succession. One example of this could be the testing of urine in one part of the device and the testing of whole blood in another part of the device. Embodiments disclosed herein may also be configured to perform a panel of biomarker tests where each biomarker is related to a specific disease state. For example, a biosensor panel may be designed for cardiac biomarkers that include choline and other species. The results from the different biomarkers of the panel may be a better prognosticator for the disease and patient outcome than just a single cardiac biomarker.

In certain examples, the electrodes may also be in fluid communication with a molecular imprinted polymer (MIP) for analyte selectivity. In certain examples, the MIP may be effective to immobilize or capture a selected biological recognition element on a surface, e.g., a surface of a working electrode. In an illustrative MIP synthesis, the target (or template) molecule may be allowed to interact with a functional monomer in a predetermined orientation. The monomer-template interaction can be reversible covalent bonding, non-covalent or metal ion coordination or other physical interactions. This monomer-template complex may then be copolymerized with a crosslinker, leading to a highly cross-linked macroporous polymer with the imprint molecules in a sterically fixed arrangement. After removal of the template molecules, recognition sites that bind specifically to the target molecules may be established.

In accordance with certain examples, the device may also include various other elements that may be used to facilitate detection of a biomarker. For example, a binder may be used to aid in forming a film, a wetting agent may be used, and one or more polymeric components may be employed to diminish or eliminate fouling of the electrode (e.g., polyethylene glycol or poly-hydroxyethylmethacrylate). In some examples, one or more cationic and anionic exchange elements may be present to remove interfering species. In addition, the device may include an electrochemical mediator to facilitate electron transfer to the working electrode. In some embodiments, size exclusion media or other filters may be used to remove species above a certain size from the sample and pass species below a certain size for detection. Other features to aid in detection of a biomarker using the devices disclosed herein will be recognized by the person of ordinary skill in the art, given the benefit of this disclosure.

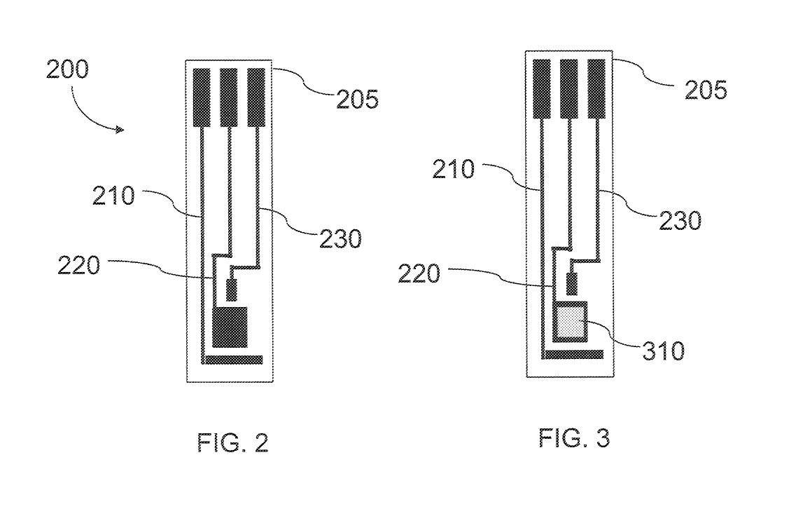

In accordance with certain examples, the devices disclosed herein may include three or more electrodes. Referring to FIG. 2, a device 200 includes three electrodes 210, 220 and 230 on an insulating support 205. In this example, the reference electrode is shown as electrode 210, the working electrode is shown as electrode 220 and an auxiliary/fill electrode is shown as electrode 230. Variations of the configuration shown in FIG. 2 may be incorporated to achieve different layout, electrode dimensions, overall sensor size, varying sample introduction methods, and ways of transferring the sample to the working (or test) electrode. For example, the electrode layout can be a variant of the pattern shown in FIG. 2 and may be constructed from a variety of conductive materials suitable for electrochemical application, including, but not limited to, gold, platinum, carbon, etc.

In accordance with certain examples, an active reagent may be brought into fluid communication with the working electrode. Referring now to FIG. 3, an active reagent 310, such as a biological recognition element, has been disposed on the working electrode 220. The reagent may be disposed on electrode(s) without the active ingredient in order to correct for background signal, e.g., buffer may be used to obtain a background signal. Methods used for disposition of the reagent mixture may include wicking by capillary action, screen printing, drop-coating, spray-coating, dip-coating, manual dispensing and/or combinations thereof, among others. Components of the reagent may simply be mechanically mixed, may be covalently linked to each other or to the electrode surface, or positioned through other physicochemical means such as electrostatic interaction or self-assembly with each other or the electrode surface. It will be within the ability of the person of ordinary skill in the art, given the benefit of this disclosure, to select suitable methods for disposing the reagents on a working electrode.

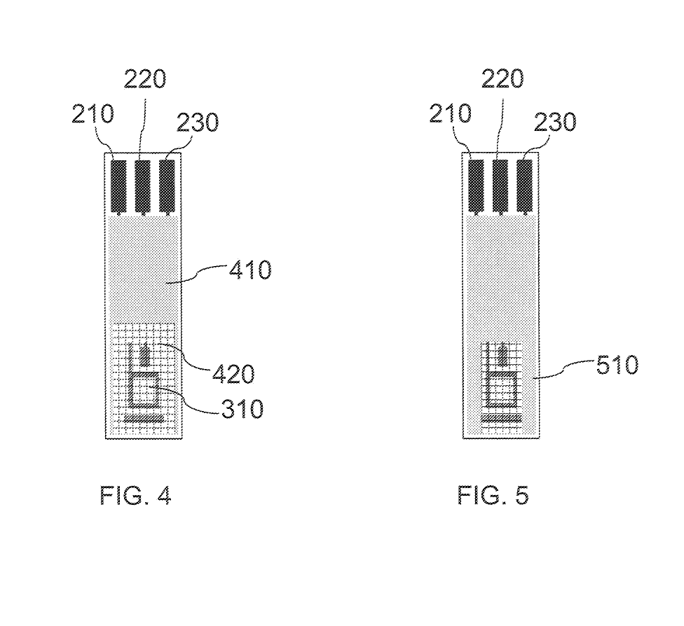

In accordance with certain examples, one or more insulating layers may be disposed on the support. Referring now to FIG. 4, deposition of an insulating layer 410, which defines a sample test area (electrochemical cell) and electrical contacts, is shown. Deposition of the insulating layer 410 may include techniques similar to those used in deposition of electrodes on the insulating substrate. In addition, FIG. 4 also shows a sample transfer layer 420 deposited to aid in transferring the test sample to the sample test area. The sample transfer layer 420 may contain certain materials, e.g., surfactant-coated materials such as polymer sheets, perforated sheets, meshes, and or combinations thereof, and may generally be configured to function as a wicking device. Illustrative materials for use as an insulating layer 410 include, but are not limited to, Polyplast PY (screen inks for plastics), silicon nitride and silicon dioxide. Illustrative materials for use as a sample transfer layer 420 include, but are not limited to, hydrophilic polyester film (3M), polyester mesh coated with a surfactant such as 3M's FC-170 and inkjet transparencies. Additional materials will be readily selected by the person of ordinary skill in the art, given the benefit of this disclosure.

In accordance with certain examples, one or more additional layers may be disposed on the support and on the insulating layer 410 and/or layer 420. Referring now to FIG. 5, a subsequent insulation layer 510 may be deposited on insulation layer 410 to improve adhesion of the sample transfer layer 420. The insulation layer 510 may be deposited using methods similar to those used to deposit insulation layer 410. The insulation layer 510 may also include materials similar to those used in the insulation layer 410. Illustrative materials for use as an insulating layer 510 include, but are not limited to, Polyplast PY, silicon nitride and silicon dioxide. Additional materials will be readily selected by the person of ordinary skill in the art, given the benefit of this disclosure.

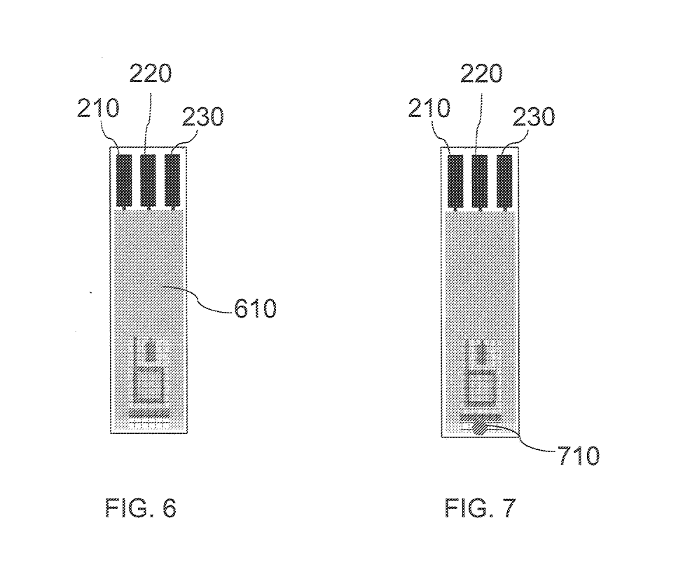

In accordance with certain examples, a protective or top layer may be disposed on the support. Referring now to FIG. 6, deposition of a protective layer 610 to protect the underlying layers is shown. The protective layer 610 may be made of a transparent or opaque layer that may or may not be coated on the inside with a material, such as, for example, a surfactant, detergent, micelles, etc. Illustrative materials for use as a protective layer 610 include, but are not limited to, polyester, PET and Mylar®. Additional materials will be readily selected by the person of ordinary skill in the art, given the benefit of this disclosure.

In accordance with certain examples, the device illustrated in FIGS. 2-6 may be used to determine the level of a biomarker in a patient sample, such as blood, urine, sweat or other body fluids. Referring now to FIG. 7, a sample 710 may be introduced into the device for performing an analysis. In certain examples, the sample may be introduced from the side, along an edge or through a hole in the top layer. One or more components in the sample, e.g., choline, may be converted by a biological recognition element to a detectable product, e.g., hydrogen peroxide. The detectable product may then be detected amperometrically, potentiometrically or by other detection methods depending on the nature of the species to be detected. In the case of electrochemical detection, the current or voltage that is measured may be compared with a current or voltage from a standard curve to determine the level of biomarker present in the sample. The current or voltage may then be displayed or outputted to a desired device, e.g., a display screen, printer, e-mail or the like. In certain examples, the current or voltage may also be converted to analyte concentration using the calibration or standard curve.

It will be recognized by the person of ordinary skill in the art, given the benefit of this disclosure, that the devices disclosed herein may include two, three, four or more electrodes. For example, an additional electrode may be used for background correction. In this configuration, a fourth electrode may include a reagent mixture without the active ingredient. Other configurations of devices that include a plurality of electrodes will be selected by the person of ordinary skill in the art, given the benefit of this disclosure.

In accordance with certain examples, the sample transfer layer may be eliminated and the protective layer may be instead coated on the inside with a suitable material, e.g., a surfactant, to permit for sample transfer into the electrochemical cell. Alternatively, the sample transfer layer may be constructed of a material to remove cells, or other selected materials, from the sample prior to reaching the test area.

In accordance with certain examples, calibration of the device may be carried out by a variety of methods including, but not limited to, entering a code provided with the device or by inserting a test strip or sample containing the calibration information for a given lot of devices. In another embodiment, the calibration information may be bar coded, for example on the container for the test strips.

In accordance with certain examples, the device may be used with whole blood, lysed blood, blood plasma/serum, cerebrospinal fluid, interstitial fluid, urine, sweat, saliva or other bodily fluid for determination of the total level of a biomarker. In some examples, the intracellular and extracellular levels of the biomarker may be detected separately by isolating the cells and then measuring the biomarker levels within the cell. Cells may be isolated using conventional techniques, such as, for example, centrifugation, pelletization and the like.

In other embodiments, the device may be adapted for micro- or nano-sensing applications, either in vivo or in vitro. For example, the device may be miniaturized and placed in a catheter (e.g., bladder catheter, kidney catheter, intravenous catheter, etc.) in a vein, artery, duct or the like and can provide real time measurements of biomarkers in a particular fluid. In certain embodiments, the device may be part of a multi-analyte system where many elements as described above may be constructed with different biological recognition elements specific to at least one other biomarker. Typical examples of specific biological recognition elements include, but are not limited to, organic ion exchangers or chelating agents, ionophores, and antibodies.

Certain specific examples are described below to facilitate a better understanding of the novel features, aspects and embodiments disclosed herein.

### EXAMPLE 1

The following is a prophetic example of determination of WBCHO by a single use, disposable POC biosensor using an electrochemical mediator. A disposable biosensor for the detection of choline in a whole blood sample may be produced by the following procedure. Refer to FIG. 2 for the biosensor components. A sheet of 10 mil polyester film (Dupont Melinex 7305) is screen-printed with Ag/AgCl ink (Ercon, Inc., Wareham, Mass.) to form both a reference electrode (210) and an auxiliary electrode (230). A second screen-printed layer using a carbon ink (Gwent Electronic Materials, UK, Carbon Ink C2000802D2) forms the base of the working electrode (220) and covers the electrical leads for all three electrodes. The shape, size and configuration of the three electrodes may conform to that as shown in FIG. 2. A third screen-printed layer of an insulating ink (DuPont #5018 UV curable dielectric) is added to delineate the electrodes and cover the electrode leads (410) as shown in FIG. 4 (without the mesh).

By means of screen-printing, 10 μL of an enzyme-mediator solution may be applied to just the working electrode (310) as shown in FIG. 3. The enzyme-mediator solution may include about 2 to 5 active units of stabilized choline oxidase (Applied Enzyme Technology, Ltd. Gwent, UK) and approximately 0.5 mg potassium ferricyanide (Sigma-Aldrich, Co.) or other applicable mediator all in a millimolar phosphate buffer solution or other similar buffer. The enzyme reagent may also include stabilizers, binders and wetting agents to allow for proper flow of the reagent in the screen-printing process. The reagent solution is dried on the electrode strip in a linear oven maintained at a temperature of about 30° C. to 35° C. A spacer laminate (ARcare 7840 Adhesives Research, Inc.) with pressure adhesive on both sides containing a longitudinal channel is placed on the electrode sensor strip so that the channel includes all three electrodes. On top of this assembly is placed a lid (ARflow 90128, Adhesives Research, Inc.) which may include a hole or port for placement of the whole blood sample at one end and a vent hole or port at the other end of the channel formed in the spacer layer as shown in FIG. 6. The lid material may include a hydrophilic coating that aids the transport of blood through the channel. The lid material may also be clear so that the blood sample can be readily observed in the sensor strip. Individual sensor strips may be cut from a sheet that contains multiple sensors. A typical method of cutting individual sensors is by using a steel-rule die.

The sensor is used by applying a drop or two of whole blood to the inlet hole of the sensor as indicated in FIG. 7. The blood sample flows by capillary action through the channel in the sensor covering all three electrodes. The sensor electrodes are connected to a detector which consists of electronics capable of measuring the current flow in the sensor as a result of the detection of the choline via the choline oxidase and mediator. The detector is configured to display the amount of choline detected by applying suitable algorithms and calibration curves to the measured current.

### EXAMPLE 2

The following prophetic example describes determination of WBCHO by a disposable biosensor containing choline oxidase without a mediator. A biosensor that is capable of detecting and measuring the amount of choline in a whole blood sample by a “mediatorless” enzyme system may be fabricated by the following method. A sheet of 10 mil polyester film (Dupont Melinex 7305) is screen-printed with Ag/AgCl ink (Ercon, Inc. Wareham, Mass.) to form both a reference electrode (210) and an auxiliary electrode (230). A second screen-printed layer using a platinized carbon ink (DuPont Microcircuit Materials #BQ321 conductive composition) forms the base of the working electrode (220). The shape, size and configuration of the three electrodes may generally conform to that as shown in FIG. 2. The rest of the physical fabrication of the mediatorless sensor is similar to that of the mediated sensor as described in EXAMPLE 1. However, the reagent for the mediatorless formulation does not contain the mediator (potassium ferricyanide). In this embodiment, the working electrode with the platinum containing screen-printed ink is able to directly detect the hydrogen peroxide formed from the reaction of choline with choline oxidase. The measurement of the resulting current may be performed using methods similar to those described in Example 1.

### EXAMPLE 3

The following prophetic example describes determination of bilirubin in whole blood by a biosensor including bilirubin oxidase with a mediator. A biosensor that can detect and measure the amount of bilirubin in a whole blood sample could be fabricated. The bilirubin biosensor may be produced by following the procedure described in Example 2. However, in place of the choline oxidase reagent solution a bilirubin oxidase solution is deposited on the working electrode (310) either by means of screen-printing or pipette dispensing. The bilirubin oxidase solution consists of approximately 2 units of Myrothecium verrucaria bilirubin oxidase (Sigma Aldrich Co) and 0.5 mg of potassium ferricyanide in a pH 8.4 phosphate buffer solution or other buffers. Once fabricated the biosensor is used to detect bilirubin by placing one to two drops of whole blood taken from a patient and placing on the inlet port of the sensor. The current from the catalysis of the bilirubin by the bilirubin oxidase may be measured by the detector in a similar manner as described in Example 1.

### EXAMPLE 4

The level of WBCHO was determined by reversed-phase liquid chromatography using a post-column enzyme reactor and electrochemical detection (LC-EC). Whole blood samples were drawn into chilled Vacutainer™ tubes containing EDTA. Samples were kept on wet ice. The collected whole blood samples were prepared as follows: 100 μL of whole blood was pipetted or into a 2 mL micro-centrifuge tube. To this fluid was added 500 μL of a dilute solution of perchloric acid to precipitate the proteins in the blood sample. The tube was capped and vortexed for 10 seconds. The tube was then centrifuged at 10,000 g for 10 minutes followed by transferring 200 μL of the supernatant into a glass autosampler vial. To this fluid was added 800 μL of a buffer solution. Aliquots of 10 μL were then injected onto the LC-EC system.

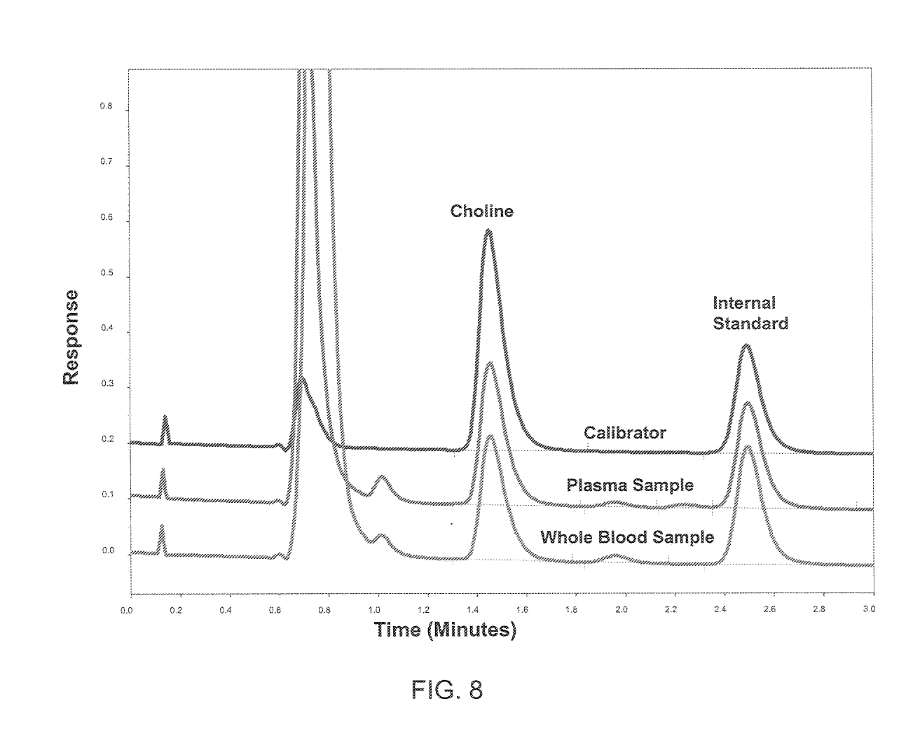

The high performance LC-EC system consisted of an autosampler (Model 542), pump (Model 584), column oven (Coulochem III Thermal Organizer) and electrochemical detector (Model 5300 Coulochem III Detector) all from ESA Biosciences, Inc. and a data chromatographic system (EZChrom SI Chromatography Data System, Scientific Software Inc.). The electrochemical cell used for the detection of the analyte consisted of a platinum working electrode and two other electrodes—a palladium reference electrode and a palladium counter or auxiliary electrode (Model 5040 Electrochemical Cell, ESA Biosciences). Directly after the autosampler and prior to the electrochemical flow cell was placed a reverse phase column (Choline Analytical Column, ESA Biosciences) and then a choline oxidase enzyme reactor column (Choline IMER, ESA Biosciences) in series. Both columns were placed into the column oven and maintained at 37° C. The sample was eluted through the HPLC system with a mobile phase consisting of a phosphate buffer containing an ion pairing reagent (octanesulfonic acid) at a flow rate of 0.5 mL/min. The working potential of the platinum working electrode was maintained at 300 mV. Chromatograms showing a prominent peak for the choline response in whole blood and plasma are shown in FIG. 8.

When introducing elements of the examples disclosed herein, the articles “a,” “an,” “the” and “said” are intended to mean that there are one or more of the elements. The terms “comprising,” “including” and “having” are intended to be open ended and mean that there may be additional elements other than the listed elements. It will be recognized by the person of ordinary skill in the art, given the benefit of this disclosure, that various components of the examples can be interchanged or substituted with various components in other examples. Should the meaning of the terms of any of the patents, patent applications or publications referred to herein conflict with the meaning of the terms used in this disclosure, the meaning of the terms in this disclosure are intended to be controlling.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

What is claimed is:

1. A device comprising:
a support;
a first electrode disposed on the support;
a second electrode disposed on the support; and
a chamber disposed on the support and comprising a sample area configured to receive a biomarker and a biological recognition element specific for the biomarker, the chamber being in fluid communication with at least one of the first electrode and the second electrode.

2. The device of claim 1, further comprising a detector electrically coupled to at least one of the first electrode and the second electrode.

3. The device of claim 1, in which the biological recognition element is an oxidoreductase.

4. The device of claim 1, in which the biomarker is a substrate and the biological recognition element is an enzyme specific for the substrate.

5. The device of claim 2, further comprising a third electrode electrically coupled to the detector.

6. The device of claim 2, in which the detector is an electrochemical detector.

7. A device comprising a support and a biological recognition element disposed on the support, the biological recognition element effective to produce an electrochemically detectable reaction product from a body fluid comprising one or more biomarkers indicative of a disease state.

8. The device of claim 7, in which the biological recognition element is selected from the group consisting of an enzyme, an antibody and an antigen.

9. The device of claim 7, in which the biological recognition element is an oxidoreductase.

10. The device of claim 7, further comprising at least one electrode for detecting the electrochemically detectable reaction product.

11. The device of claim 7, further comprising an electrode array for detecting the electrochemically detectable reaction product.

12. The device of claim 7, in which the device is configured to detect the electrochemically detectable reaction product when the biomarker is present above a threshold value in the body fluid.

13. The device of claim 7, in which at least one of the one or more biomarkers is a substrate and the biological recognition element is an enzyme specific for the substrate.

14. A point of care device for detecting a biomarker indicative of a disease state, the device configured to receive a body fluid and comprising a biological recognition element effective to convert a biomarker in the body fluid into an electrochemically detectable reaction product.

15. The point of care device of claim 14, in which the biological recognition element is an oxidoreductase.

16. The point of care device of claim 14, further comprising an electrochemical detector for detecting the electrochemically detectable reaction product.

17. The point of care device of claim 16, in which the electrochemical detector is configured for potentiometric, coulometric or charged aerosol detection.

18. A method of detecting a biomarker in a body fluid, the method comprising exposing the biomarker to a biological recognition element disposed in a device comprising at least one electrode, and detecting a reaction product after conversion of the biomarker into the reaction product by the biological recognition element.

19. The method of claim 18, in which the detecting step comprises electrochemically detecting the reaction product.

20. The method of claim 18, further comprising detecting a second reaction product after conversion of a second biomarker in the body fluid into the second reaction product by a second biological recognition element disposed in the device.
